(19) United States
(12) Patent Application Publication
(10) Pub. No.: US 2015/0142370 A1
(43) Pub. Date: May 21, 2015

(54) TRACKER AND OPERATION PROCESS THEREOF

(71) Applicant: CHUN-YI LEU, Taipei City (TW)

(72) Inventor: CHUN-YI LEU, Taipei City (TW)

(21) Appl. No.: 14/148,701

(22) Filed: Jan. 6, 2014

(30) Foreign Application Priority Data
Nov. 15, 2013 (TW) 102141792

(51) Int. Cl. A61B 5/11; A61B 5/00; G01C 22/00; G01C 23/00; G01P 13/00; G01P 15/00

(52) U.S. Cl. CPC A61B 5/1123; G01P 13/00; G01P 15/00; G01C 22/006; G01C 23/00; A61B 5/1118; A61B 5/6828
USPC 702/141

(57) ABSTRACT

The present invention provides a tracker and the operation process thereof. In one aspect, the tracker, designed for positioning on a part below a user's knee, provided in the present invention is able to switch between modes such as walking mode, cycling mode, swimming mode, sleeping mode, and other modes by detecting the motions of a user. By the sensing unit included in the tracker, the tracker can further detect signatures, such as steps taken, distance traveled, stairs climbed, calories burned, cycling speed, swimming type, or heart rate, of a user. Furthermore, the tracker in the present invention is able to synchronize or stream information with other devices. In another aspect, the tracker in the present invention is able to control avatars of a game on other devices as a sensory tool.

### TECHNICAL FIELD

The present invention relates to a tracker and the operation process thereof. More particularly, the invention relates to a multifunctional tracker capable of supporting several operating modes and the operation process thereof.

### BACKGROUND ART

Obesity has recently become the leading threaten of health worldwide, mainly because of changes in lifestyle and diet nowadays. With increasing prevalence of obesity, which induces various cardiovascular diseases, type 2 diabetes, and even certain types of cancer in population, authorities has started emphasizing the importance of weight control. According to medical researches and nutritionists, adequate exercise is considered as an appropriate way of weight control instead of dieting. However, the ideal quantity of exercise varies depending on factors such as gender, age, smoking, gene, and body mass index (BMI). To quantify exercise intensity, people usually use sensing devices while exercising to gather numeric information, and further optimize exercise plans by that information.

One of the most common sensing devices is pedometer. A pedometer was positioned about a user's hip and counts each step the user takes by detecting the motion. An advanced pedometer also monitors distance, velocity, heart rate, and calorie burned of a user. Another example of a sensing device is sport watch. Some sport watches are capable of monitoring distance, duration, heart rate, and calorie burned of an exercise; they are also capable of operating under different situations including walking, running, cycling, or marathon.

With development of new technologies, the size and the shape of these sensing devices are greatly varied and mostly were designed to a watch-like style to be easily carried with a user. However, functions of these sensing devices are limited. A pedometer is only suitable for walking and running, which may not satisfy the demands from people with active lifestyle. A sport watch is compatible with several different exercises, but the interaction is restricted between the user and the sport watch. As a result, a user may refrain from experiencing all the benefits of the sensing device.

### SUMMARY OF INVENTION

The present invention provides a tracker designed for positioning on a part below the user's knee, wherein the tracker comprising a processing unit, including at least one operating mode, for generating a feedback signal; a sensing unit, connected to the first processing unit, for detecting movements and generating an acceleration signal or a motion signal provided for the first processing unit; a memory unit, connected to the first processing unit, for storing the feedback signal; and a user interface, connected to the first processing unit, for displaying the feedback signal. The tracker in the present invention is invented firstly to reach a variety of demands from people with active lifestyle, secondly to stream information to other devices, and thirdly to control the avatars of a game on other devices as a sensory tool.

The tracker provided in this invention, wherein the sensing unit is one selected from the group consisting of a gyroscope, an accelerometer, and an infrared sensor; the at least one operating mode is one selected from the group consisting of basic mode, walking mode, cycling mode, sleeping mode, swimming mode, and synchronization mode; the motion signal is one selected from the group consisting of a walking signal, a stair-climbing signal, a cycling signal, a sleeping signal, and a swimming signal; the feedback signal is one selected from the group consisting of steps taken, distance traveled, stairs climbed, calories burned, cycling speed, swimming type, and heart rate.

The tracker provided in the present invention further comprises a wireless transceiver, connected to the first processing unit, for transmitting the feedback signal to a receiving device via a wireless network. The wireless transceiver here is one selected from the group consisting of a WiFi transceiver, a Bluetooth transceiver, and a near-field communication (NFC) transceiver. The receiving device mentioned above comprises a control unit, a second processing unit, and a display; the control unit, connected to the second processing unit, receives the feedback signal and transmits the feedback signal to the second processing unit; and the second processing unit, further connected to the display, transmits the feedback signal to the display. Wherein the receiving device is one selected from the group consisting of a mobile phone, a tablet, an LCD TV, a screen, and a display.

The present invention also provides an operation process of tracker comprising the steps of: (A) a sensing unit which has detected a movement generates an acceleration signal; (B) a first processing unit which has received the acceleration signal switches at least one operating mode; (C) the sensing unit which has detected a movement generates a motion signal; (D) the first processing unit which has received the motion signal generates a feedback signal and stores the feedback signal into a memory unit; the first processing unit further displays the feedback signal via a user interface; (E) a wireless transceiver connected to a wireless network transmits the feedback signal stored in the memory unit to a receiving device; (F) a control unit comprised in the receiving device receives the feedback signal and transmits the feedback signal to a second processing unit; (G) the second processing unit received the feedback signal displays the feedback signal via a display.

In one aspect, the tracker herein is therefore able to reach a variety of demands from people with active lifestyle. The tracker herein is capable of switching between modes such as basic mode, walking mode, cycling mode, sleeping mode, swimming mode, or synchronization mode; furthermore, by detecting the motions of a user, the tracker is capable of determining steps taken, distance traveled, stairs climbed, calories burned, cycling speed, swimming type, or heart rate. In another aspect, the tracker herein is able to stream information to other devices via a controller. In still another aspect, the tracker herein is able to control the avatars of a game on other devices as a sensory tool.

### BRIEF DESCRIPTION OF DRAWINGS

FIG. 1 illustrates a block diagram of components of an example tracker in accordance with the present invention.

FIG. 2 is a flow diagram of example operation process in accordance with the present invention.

FIG. 3 is a schematic flow diagram of manually switching between modes of an example operation process in accordance with the present invention.

FIG. 4 is a detailed flow diagram of manually switching between modes of an example operation process in accordance with the present invention.

FIG. 5(a) illustrated a diagram of signals generated by an example gyroscope in accordance with the present invention under walking

FIG. 5(b) illustrated a diagram of signals generated by an example gyroscope in accordance with the present invention under stair-climbing.

FIG. 5(c) illustrated a diagram of signals generated by an example gyroscope in accordance with the present invention under cycling.

FIG. 6(a) illustrated a diagram of signals generated by an example accelerometer in accordance with the present invention under cycling.

FIG. 6(b) illustrated a diagram of signals generated by an example accelerometer in accordance with the present invention under walking

FIG. 7 is a schematic flow diagram of automatically switching between modes of an example operation process in accordance with the present invention.

FIG. 8 is a detailed flow diagram of automatically switching between modes of an example operation process in accordance with the present invention.

### DESCRIPTION OF EMBODIMENTS

In a general aspect, the present invention relates to a tracker and the operation process thereof. More particularly, the invention relates to a multifunctional tracker, designed for positioning on a part below the user's knee, capable of supporting several operating modes and the operation process thereof. The embodiments and drawings provided here show different aspects of the present invention. However, the present invention are neither limited to any embodiment nor drawing thereof.

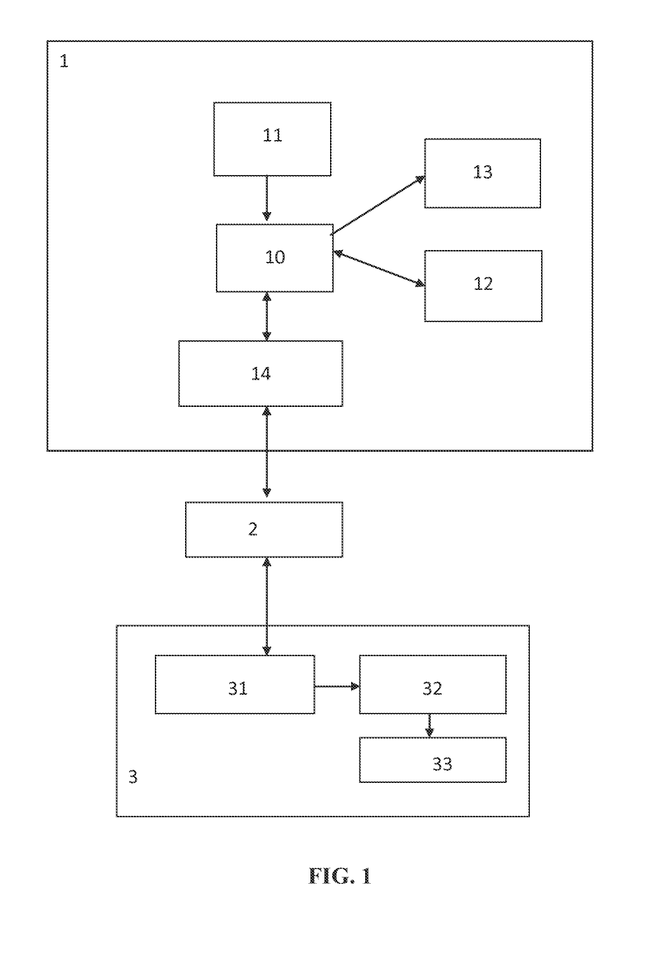

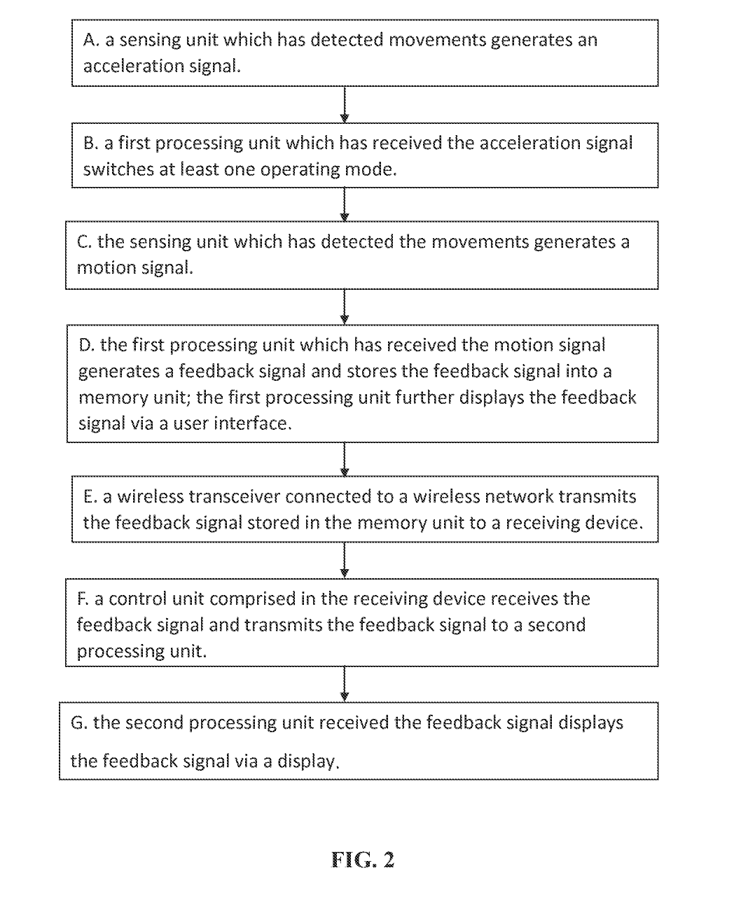

In one embodiment, as shown in FIG. 1, the tracker 1 of the present invention may comprise: a first processing unit 10, including at least one operating mode, for generating a feedback signal; a sensing unit 11, connected to the first processing unit 10, for detecting movements and generating an acceleration signal or a motion signal provided for the first processing unit 10; a memory unit 12, connected to the first processing unit 10, for storing the feedback signal; and a user interface 13, connected to the first processing unit 10, for displaying the feedback signal. The tracker 1 may comprise a wireless transceiver 14, connected to the processing unit 10, for transmitting the feedback signal to a receiving device 3 via a wireless network 2.

The receiving device 3 may comprises a control unit 31, a second processing unit 32, and a display 33; the control unit 31, connected to the second processing unit 32, receives the feedback signal and transmits the feedback signal to the second processing unit 32; and the second processing unit 32, further connected to the display 33, transmits the feedback signal to the display 33.

The sensing unit 11 may be one selected from the group consisting of a gyroscope, an accelerometer, and an infrared sensor. The at least one operating mode may be one selected from the group consisting of basic mode, walking mode, cycling mode, sleeping mode, swimming mode, and synchronization mode. The motion signal may be one selected from the group consisting of a walking signal, a stair-climbing signal, a cycling signal, a sleeping signal, and a swimming signal. The feedback signal may be one selected from the group consisting of steps taken, distance traveled, stairs climbed, calories burned, cycling speed, swimming type, and heart rate. The receiving device 3 may be one selected from the group consisting of a mobile phone, a tablet, an LCD TV, a screen, and a display. The wireless transceiver 14 may be one selected from the group consisting of a WiFi transceiver, a Bluetooth transceiver, and a near-field communication (NFC) transceiver; the wireless transceiver 14 may transmit the feedback signal via the wireless network 2, wherein the wireless network 2 may be one selected from the group consisting of a WiFi network, a Bluetooth network, and a NFC network.

The heart rate mentioned here may be detected by an infrared sensor, wherein the infrared sensor is able to detect an infrared light which has penetrated a vessel and been reflected by bones. Due to the reason that the pulsing of blood flow in the vessel may interfere the penetration of infrared light, the infrared sensor may detect the variation of infrared light and pass this information to the processing unit 10 for computing. The processing unit 10, then, computes the heart rate based on this information.

Since the tracker 1 is able to detect movements and generate signals, the tracker 1 may evaluate the exercise intensity of a user is sufficient or insufficient. Furthermore, since the tracker 1 is able to detect movements and generate feedback signals, the tracker 1 may be utilized as a sensory tool to control avatars of a game on the receiving device 3.

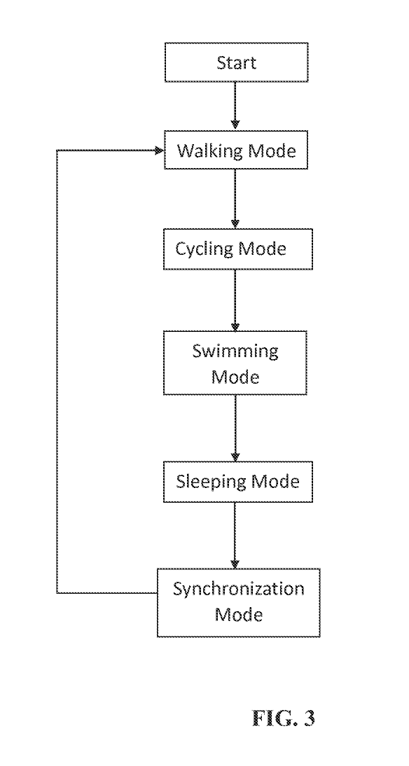

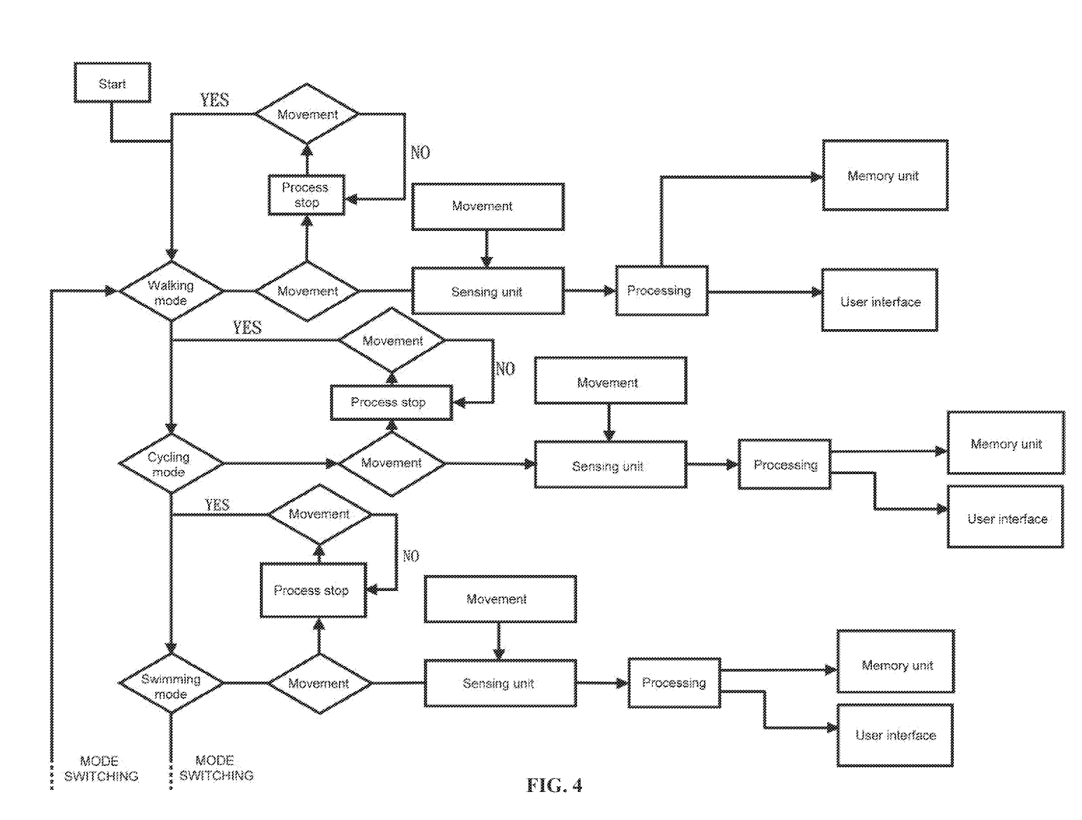

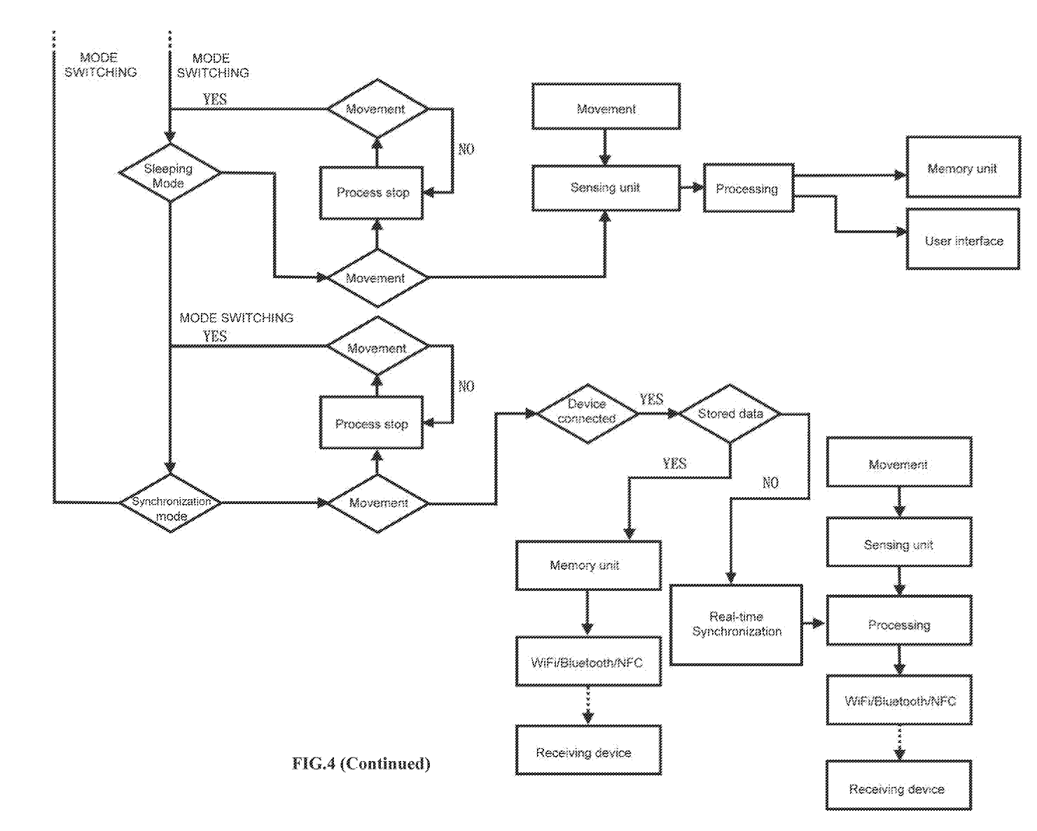

FIGS. 2-4 illustrate an operation process of tracker 1 in another embodiment in accordance with the present invention. FIG. 2 illustrates a flow diagram of the operation process of tracker 1; FIG. 3 illustrates a schematic flow diagram of manually switching; and, FIG. 4, on the contrary, further illustrates a detailed flow diagram of manually switching. In FIGS. 3 and 4, squares in the diagrams represent processing points; diamonds in the diagrams represent check-and-decision points. The operation process of tracker comprising the steps of: A. a sensing unit 11 which has detected movements generates an acceleration signal; B. a first processing unit 10 which has received the acceleration signal switches at least one operating mode; C. the sensing unit 11 which has detected the movements generates a motion signal; D. the first processing unit 10 which has received the motion signal generates a feedback signal and stores the feedback signal into a memory unit 12; the first processing unit 10 further displays the feedback signal via a user interface 13; E. a wireless transceiver 14 connected to a wireless network 2 transmits the feedback signal stored in the memory unit 12 to a receiving device 3; F. a control unit 31 comprised in the receiving device 3 receives the feedback signal and transmits the feedback signal to a second processing unit 32; G. the second processing unit 32 received the feedback signal displays the feedback signal via a display 33.

Once a user taps the tracker 1 to initiate the mode switching process, the sensor unit 11 starts to detect movements and generates acceleration signals to the first processing unit 10; the first processing unit 10 switches the operation mode to basic mode, walking mode, cycling mode, sleeping mode, swimming mode, or synchronization mode based on the acceleration signals. If there is no movement detected by the sensing unit 11 in a period of time, the operation process of tracker 1 stops processing automatically; if the sensing unit 11 detects movements and generates motion signals, such as a walking signal, a stair-climbing signal, a cycling signal, a sleeping signal, and a swimming signal, to the first processing unit 10; the first processing unit then converts the motion signals into feedback signals, such as steps taken, distance traveled, stairs climbed, calories burned, cycling speed, swimming type, or heart rate, and stores the feedback signals into the memory unit 12; the first processing unit may further display the feedback signals on a user interface 13.

The sensing unit 11 may be one selected from the group consisting of a gyroscope, an accelerometer, and an infrared sensor. The heart rate mentioned here may be detected by an infrared sensor, wherein the infrared sensor is able to detect an infrared light which has penetrated a vessel and been reflected by bones. Due to the reason that the pulsing of blood flow in the vessel may interfere the penetration of infrared light, the infrared sensor may detect the variation of infrared light and pass this information to the processing unit 10 for computing. The processing unit 10, then, computes the heart rate based on this information.

To display the feedback signal on a receiving device 3, the operation processing of tracker 1 is switched to synchronization mode and starts streaming the feedback signal to the receiving device 3. Under synchronization mode, the tracker 1 sends the feedback signal stored in the memory unit 12 to the receiving device 3 via the wireless network 2 by a wireless transceiver 14; wherein the wireless transceiver 14, connected to the memory unit 12, may be one selected from the group consisting of a WiFi transceiver, a Bluetooth transceiver, and a near-field communication (NFC) transceiver. Once the second processing unit 32 in the receiving device 3 receives the feedback signal, the second processing unit 32 may display the feedback signal by a display 33.

In yet another embodiment, the tracker 1 may send the feedback signals immediately after the first processing unit 10 converted the motion signals detected by the sensing unit 11 into the feedback signals, without the requirement of the memory unit 12. In this embodiment, the tracker 1 may control avatars of a game on the receiving device 3 as a sensory tool.

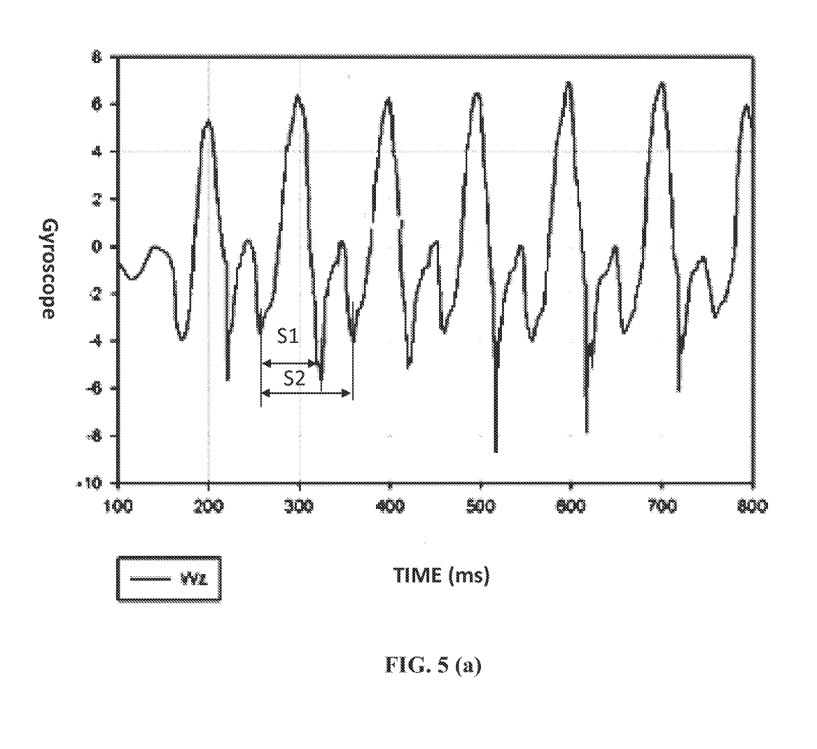

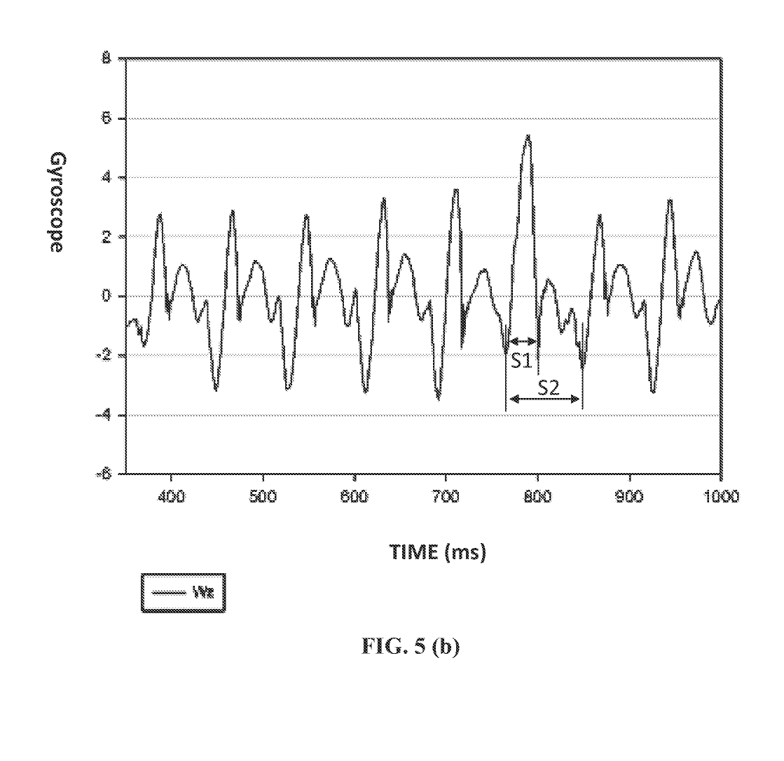

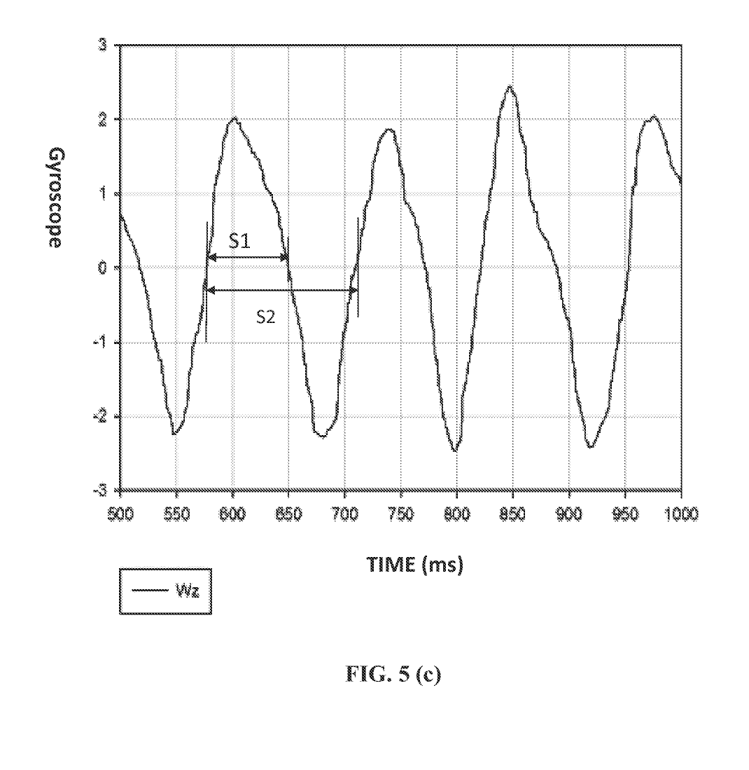

FIGS. 5(a)-(c) represent the motion signals generated by an example gyroscope in accordance with the present invention. FIG. 5(a) illustrated a diagram of signals generated by an example gyroscope under walking FIG. 5(b) illustrated a diagram of signals generated by an example gyroscope under stair-climbing. FIG. 5(c) illustrated a diagram of signals generated by an example gyroscope under cycling. Vectors (Wz) in FIGS. 5(a)-(c) shows positive while the tracker 1 is being lifted indicates that motion signals detected by the gyroscope is high fidelity; wherein S1 represents the motion signal of leg-lifting generated by the gyroscope under walking, stair-climbing, or cycling, and S2 represents an entire cycle of motion signal included leg-lifting and leg-lowering. According to the diagrams in FIGS. 5(a)-(c), the patterns generated by the gyroscope under walking, stair-climbing, or cycling are distinguishable and may be recognized easily.

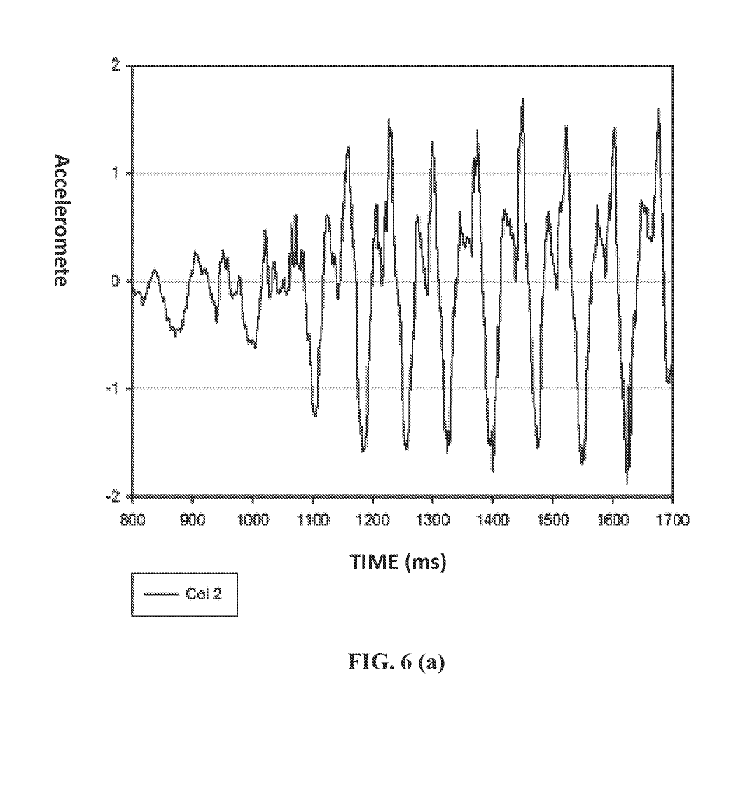

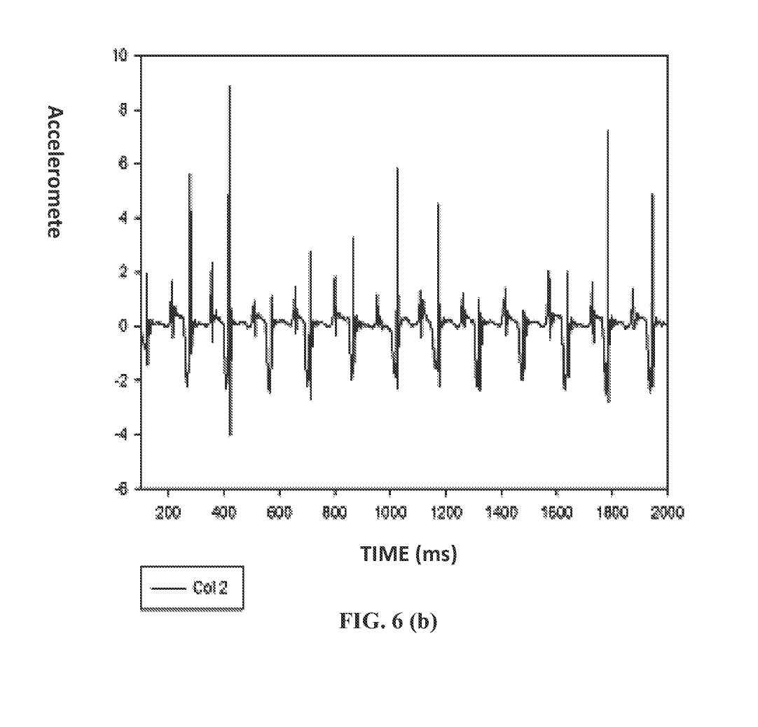

FIGS. 6(a) and (b) represent the motion signals generated by an example accelerometer in accordance with the present invention. FIG. 6(a) illustrated a diagram of signals generated by an example accelerometer under cycling. FIG. 6(b) illustrated a diagram of signals generated by an example accelerometer under walking According to the diagrams in FIGS. 6(a) and (b), the patterns generated by the accelerometer under walking or cycling are distinguishable and may be recognized easily.

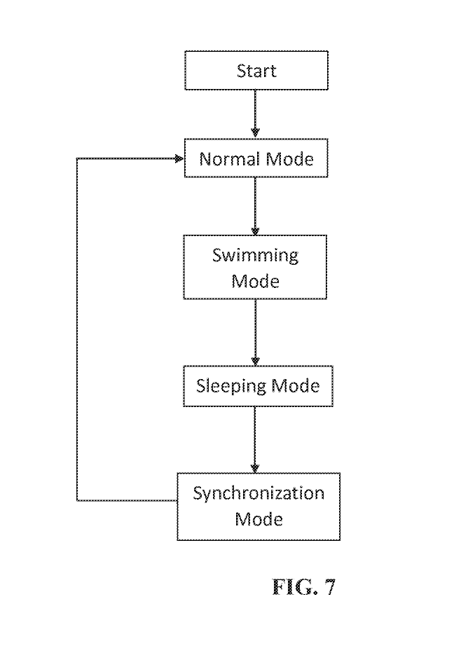

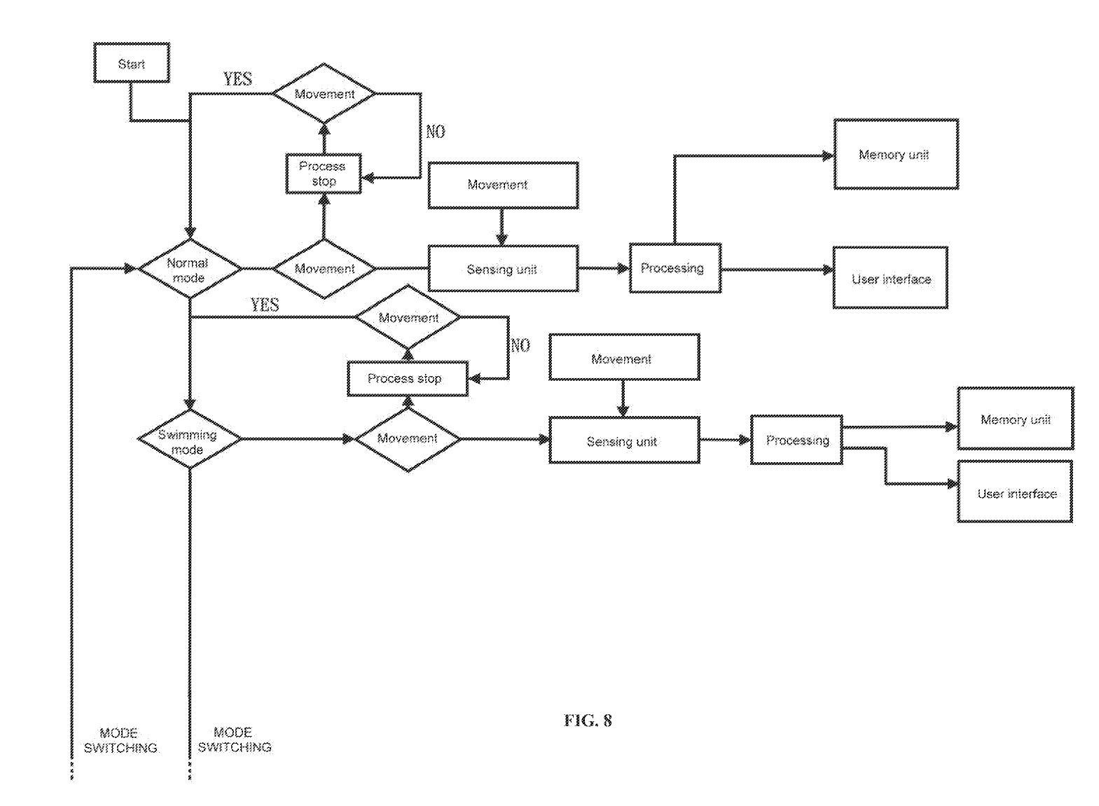

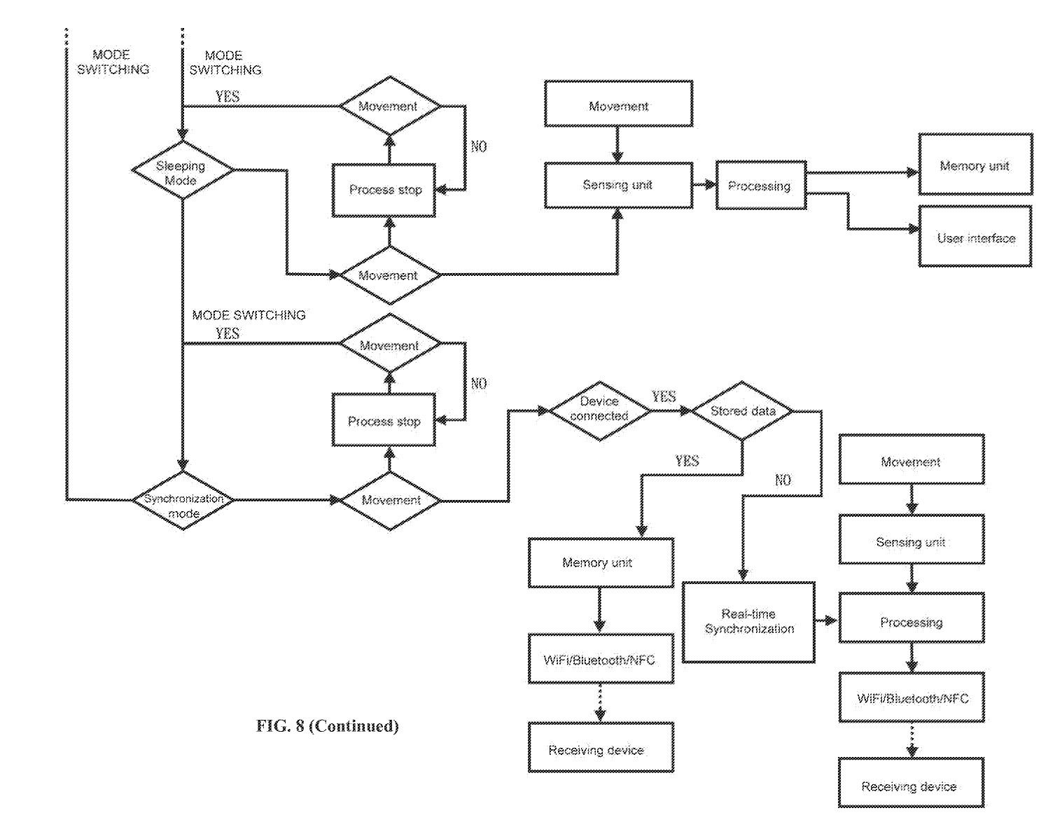

FIG. 7 illustrates a schematic flow diagram of automatically switching; and, FIG. 8, on the contrary, further illustrates a detailed flow diagram of automatically switching. Squares in the diagrams represent processing points; diamonds in the diagrams represent check-and-decision points. Under normal mode, the tracker 1 is able to recognize a user is under walking, stair-climbing, or cycling. If there is no movement detected by the sensing unit 11 in a period of time, the operation process of tracker 1 stops processing automatically; if the sensing unit 11 detected movements, the sensing unit 11 generates motion signals, such as a walking signal, a stair-climbing signal, a cycling signal, a sleeping signal, and a swimming signal, to the first processing unit 10; the first processing unit then converts the motion signals into feedback signals, such as steps taken, distance traveled, stairs climbed, calories burned, cycling speed, swimming type, or heart rate, and stores the feedback signals into the memory unit 12; the first processing unit may further display the feedback signals on a user interface 13.

To display the feedback signal on a receiving device 3, the operation processing of tracker 1 is switched to synchronization mode and starts streaming the feedback signal to the receiving device 3. Under synchronization mode, the tracker 1 sends the feedback signal stored in the memory unit 12 to the receiving device 3 via the wireless network 2 by a wireless transceiver 14; wherein the wireless transceiver 14, connected to the memory unit 12, may be one selected from the group consisting of a WiFi transceiver, a Bluetooth transceiver, and a near-field communication (NFC) transceiver. Once the second processing unit 32 in the receiving device 3 receives the feedback signal, the second processing unit 32 may display the feedback signal by a display 33.

The tracker 1 also may send the feedback signals immediately after the first processing unit 10 converted the motion signals detected by the sensing unit 11 into the feedback signals, without the requirement of the memory unit 12. In this embodiment, the tracker 1 may control avatars of a game on the receiving device 3 as a sensory tool.

To sum up, the tracker 1 in the present invention is able to switch between modes manually; the tracker 1 in the present invention is also able to recognize a user is under walking, stair-climbing, or cycling automatically. In one aspect, by the sensing unit 11 in the tracker 1, the tracker 1 is capable of detecting movements and thus computes steps taken, distance traveled, stairs climbed, calories burned, cycling speed, swimming type, or heart rate. In another aspect, the tracker 3 in the present invention is able to control avatars of a game on other devices as a sensory tool.

What is claimed is:

1. A tracker comprising:
a first processing unit, including at least one operating mode, for generating a feedback signal;
a sensing unit, connected to the first processing unit, for detecting movements and generating an acceleration signal or a motion signal provided for the first processing unit;
a memory unit, connected to the first processing unit, for storing the feedback signal; and
a user interface, connected to the first processing unit, for displaying the feedback signal.

2. The tracker according to claim 1, further comprising a wireless transceiver, connected to the first processing unit, for transmitting the feedback signal to a receiving device via a wireless network.

3. The tracker according to claim 2, wherein the receiving device is one selected from the group consisting of a mobile phone, a tablet, an LCD TV, a screen, and a display.

4. The tracker according to claim 2, wherein the wireless transceiver is one selected from the group consisting of a WiFi transceiver, a Bluetooth transceiver, and a near-field communication (NFC) transceiver.

5. The tracker according to claim 1, wherein the sensing unit is one selected from the group consisting of a gyroscope, an accelerometer, and an infrared sensor.

6. The tracker according to claim 1, wherein the at least one operating mode is one selected from the group consisting of basic mode, walking mode, cycling mode, sleeping mode, swimming mode, and synchronization mode.

7. The tracker according to claim 1, wherein the motion signal is one selected from the group consisting of a walking signal, a stair-climbing signal, a cycling signal, a sleeping signal, and a swimming signal.

8. The tracker according to claim 1, wherein the feedback signal is one selected from the group consisting of steps taken, distance traveled, stairs climbed, calories burned, cycling speed, swimming type, and heart rate.

9. The tracker according to claim 2, wherein the receiving device comprises a control unit, a second processing unit, and a display; the control unit, connected to the second processing unit, receives the feedback signal and transmits the feedback signal to the second processing unit; and the second processing unit, further connected to the display, transmits the feedback signal to the display.

10. A operation process of a tracker comprising the steps of:
A. a sensing unit which has detected movements generates an acceleration signal;
B. a first processing unit which has received the acceleration signal switches at least one operating mode;
C. the sensing unit which has detected the movements generates a motion signal;
D. the first processing unit which has received the motion signal generates a feedback signal and stores the feedback signal into a memory unit; the first processing unit further displays the feedback signal via a user interface;
E. a wireless transceiver connected to a wireless network transmits the feedback signal stored in the memory unit to a receiving device;
F. a control unit comprised in the receiving device receives the feedback signal and transmits the feedback signal to a second processing unit;
G. the second processing unit received the feedback signal displays the feedback signal via a display.

11. The operation process of a tracker according to claim 10, wherein the sensing unit is one selected from the group consisting of a gyroscope, an accelerometer, and an infrared sensor.

12. The operation process of a tracker according to claim 10, wherein the at least one operating mode is one selected from the group consisting of basic mode, walking mode, cycling mode, sleeping mode, swimming mode, and synchronization mode.

13. The operation process of a tracker according to claim 10, wherein the motion signal is one selected from the group consisting of a walking signal, a stair-climbing signal, a cycling signal, a sleeping signal, and a swimming signal.

14. The operation process of a tracker according to claim 10, wherein the feedback signal is one selected from the group consisting of steps taken, distance traveled, stairs climbed, calories burned, cycling speed, swimming type, and heart rate.

15. The operation process of a tracker according to claim 10, wherein the receiving device is one selected from the group consisting of a mobile phone, a tablet, an LCD TV, a screen, and a display.

16. The operation process of a tracker according to claim 10, wherein the wireless transceiver is one selected from the group consisting of a WiFi transceiver, a Bluetooth transceiver, and a near-field communication (NFC) transceiver.
